(19) United States
(12) Patent Application Publication
(10) Pub. No.: US 2018/0208529 A1
(43) Pub. Date: Jul. 26, 2018

(54) SERIAL METHANOL REACTORS

(71) Applicant: Haldor Topsøe A/S, Kgs. Lyngby (DK)

(72) Inventor: Mohemmad Sadath Mehaboobjan, Copenhagen (DK)

(73) Assignee: Haldor Topsøe A/S, Kgs. Lyngby (DK)

(21) Appl. No.: 15/320,235

(22) PCT Filed: Jun. 18, 2015
(86) PCT No.: PCT/EP2015/063719

§ 371 Date: Dec. 19, 2016

(30) Foreign Application Priority Data
Jun. 20, 2014 (DK) PA 2014 00327

(51) Int. Cl. C07C 29/151; C07C 31/04; B01J 8/02; B01J 8/04

(52) U.S. Cl. CPC C07C 29/1516; C07C 31/04; B01J 8/0242; B01J 8/0457; B01J 8/0492; C01B 32/40; B01J 2208/00176; B01J 2208/0053; B01J 2208/025; B01J 8/0461; B01J 8/0496

(57) ABSTRACT

The present relates to a process comprising the steps of Providing a syngas stream with module M to a Methanol loop, In the Methanol loop passing the syngas though a first Methanol reactor, obtaining a first effluent from the first Methanol reactor, Cooling the first effluent and condensing at least part of the produced methanol Separating the first cooled effluent into at least a first raw Methanol stream and a first unreacted stream, Heating the first unreacted stream, Passing the first heated unreacted stream through a second methanol reactor, Obtaining a second effluent from the second methanol reactor, Separating the second effluent into at least a second raw Methanol stream and a second unreacted stream, and Recycling the second unreacted stream to the syngas stream.

The present application relates to methanol production.

Methods for the production of methanol by catalytic conversion of synthesis gas containing hydrogen and carbon oxides have been known for a long time to persons skilled in the art.

A methanol plant with natural gas feed can be divided into three main sections. In the first part of the plant natural gas is converted into synthesis gas. The synthesis gas reacts to produce methanol in the second section, and methanol is purified to the desired purity in the tail-end of the plant. In a standard methanol synthesis loop, a reactor such as a boiling-water reactor (BWR) is used to convert the synthesis gas from a reformer/gasifier unit and recycle gas, i.e. unconverted synthesis gas into Methanol.

About the Methanol process it can generally be said that the Methanol synthesis gas is characterised by the stoichiometric ratio (H₂— CO₂)/(CO+CO₂), often referred to as the module M. A module M of 2 defines a stoichiometric synthesis gas for formation of methanol. Other important properties of the synthesis gas are the CO to CO₂ ratio and the concentration of inerts. When introduced in the Methanol loop the synthesis gas is often referred to as make-up gas. A high CO to CO₂ ratio will increase the reaction rate and the achievable per pass conversion. In addition, the formation of water will decrease, reducing the catalyst deactivation rate. High concentration of inerts will lower the partial pressure of the active reactants. Inerts in the Methanol synthesis are typically methane, argon and nitrogen.

CO₂+3H₂↔CH₃OH+H₂O

(-ΔH 298K,50Bar=40.9 kJ/mol) (1)

CO+2H₂↔CH₃OH

(-ΔH 298K,50Bar=90.7 kJ/mol) (2)

CO₂+H₂↔CO+H₂O

(-ΔH 298K,50Bar=49.8 kJ/mol) (3)

The methanol synthesis is exothermic and the maximum conversion may often be obtained at low temperature and high pressure.

The capacity of methanol plants is increasing to reduce investments, taking advantage of the economy of scale. The capacity of a world scale plant has increased from 2500 MTPD a decade ago to about 5000 MTPD today. Even larger plants up to 10,000 MTPD or above are considered to further improve economics and to provide the feedstock for the Methanol-to-Olefin (MTO) process. This constantly increases the demand on new build plants as well as the demand for optimization and revamps of existing plants is in constant focus.

Thus there is a need for new processes and plants which increase capacity and efficiency of new and existing plants.

In a first aspect of the present process and plant is provided a way to increase the conversion of the CO and CO₂ per pass and in this way increasing the carbon efficiency and/or the synthesis loop recycle gas flow rate and thus improve the energy efficiency and reduce the size of the recycle gas compressor.

In a second aspect of the present process and plant is provided a way to increase the capacity of an existing methanol plant.

These and other advantages is achieved by a process comprising the steps of
Providing a syngas stream with module M to a Methanol loop,
In the Methanol loop passing the syngas though a first Methanol reactor,
obtaining a first effluent from the first Methanol reactor,
Cooling the first effluent and condensing at least part of the produced methanol
Separating the first cooled effluent into at least a first raw Methanol stream and a first unreacted stream,
Heating the first unreacted stream,
Passing the first heated unreacted stream through a second methanol reactor,
Obtaining a second effluent from the second methanol reactor,
Cooling the second effluent and condensing at least part of the produced methanol
Separating the cooled second effluent into at least a second raw Methanol stream and a second unreacted stream, and
Recycling the second unreacted stream to the syngas stream.

Thus by the present process it is possible to improve the conversion per pass in the synthesis loop by installing one or more additional methanol reactor(s) downstream the first methanol separator in the loop.

As the methanol synthesis is restricted by equilibrium installing the reactor after the separator the product from the methanol reactions is removed and potential for further reaction of unreacted gas is obtained making it possible to have further conversion without increasing the severity of the 2nd (and subsequent) reactor(s).

The present process can be further extended by one or more separator/Methanol reactor sets. For example it is possible to have a series of First reactor-first separator-second reactor-second separator-third reactor-third separator etc. In case of more than two methanol reactors the recycle lead to mixing with the syngas is preferably taken from the last separator.

The first and second methanol reactor may be of the same or different type. E.g. the first reactor and or second reactor may be a boiling water reactor (BWR), an adiabatic reactor and/or a quench reactor.

If the first and second type is the same in form of BWR reactors it may be an advantage that the two reactors can be made identical facilitating the reactor manufacturing.

The first and second reactor may be of different types. For example if the first methanol reactor is a boiling water reactor the major part of the conversion is done in this reactor and the second methanol reactor can be a simpler type e.g. a simple adiabatic reactor with a relative smaller catalyst volume.

The catalyst in one or more of the Methanol reactors may for example be a known Methanol catalyst such as an Alumina based catalyst. Examples are 6×4 mm Cu/Zn/Al₂O₃ catalyst for example Topsøse MK-121 or MK-151 Fence™.

The first reactor and or second reactor can be operated at the same or different inlet temperatures in order to optimite the efficiency of the conversion and/or catalyst in the individual reactor.

For example the first and/or second reactor may be operated in the temperature range 150-310° C., preferably 190-280° C. and/or at pressure 50-150 kg/cm2 g, preferably 70-90 kg/cm2 g.

The first reactor and/or second reactor can depending on e.g. the type of reactors be operated with the same or different catalyst. If the second reactor is an adiabatic reactor it can be possible to utilize a more thermo-stable catalyst than used in the first reactor of a different type.

A purge can be taken preferably from the final recycle stream in order to avoid accumulation of inerts in the loop.

At least one compressor can be arranged in one or more of the unreacted streams such as in the first and/or second unreacted stream (recycle). In case the methanol loop comprises a third, fourth etc. set of Methanol reactor/separator, a compressor may alternatively/also be arranged in one or more of the unreacted stream from these.

Thus by the present process and plant, it is possible to increase the conversion of the CO and CO₂ per pass and in this way increasing the carbon efficiency and/or the synthesis loop recycle gas flow rate and thus improve the energy efficiency and reduce the size of the recycle gas compressor.

The present process can be used in new methanol production plants, and it is also usable for revamping of existing plants comprising at least one Methanol loop in order to increase the capacity of such plants.

In some setups the present process and plant may be particularly useful in relation to revamp projects. In many revamps of existing Methanol plants actions are taken to improve the module of the synthesis gas to the methanol reactor thereby increasing the molar weight of the recycle gas. Using existing compressor(s), typically centrifugal compressors supplying a constant head for same volumetric flow and compressor speed. This means that the differential pressure actually will increase when the molecular weight is increased. In this situation the present process can advantageously be applied compared to e.g. processes wherein the reactors are arranged in parallel due to the increased pressure drop over the reactors in the present system compared to increased flow in parallel reactors.

In revamps the present process method and plant arrangement is applied to one or more existing methanol loops thereby achieving an optimized plant with increased capacity.

The capacity increase can be obtained by using the present process alone, or in combination with other revamp options.

The present process may be carried out in a plant comprising one or more Methanol loops wherein at least one of the methanol loops is arranged with two or more serially connected methanol with belonging separators for separating cooled reactor effluent into unreacted gas and raw methanol. The plant may further comprise a compressor for the syngas entering the loop and one or more compressors arranged in one or more of the unreacted streams. Furthermore the plant may comprise means for heating the streams before each reactor as well as coolers and condensers for condensing the effluent from each of the reactor before entering the subsequent separator. The raw methanol streams, one from each separator, may advantageously be collected into a collected raw methanol stream.

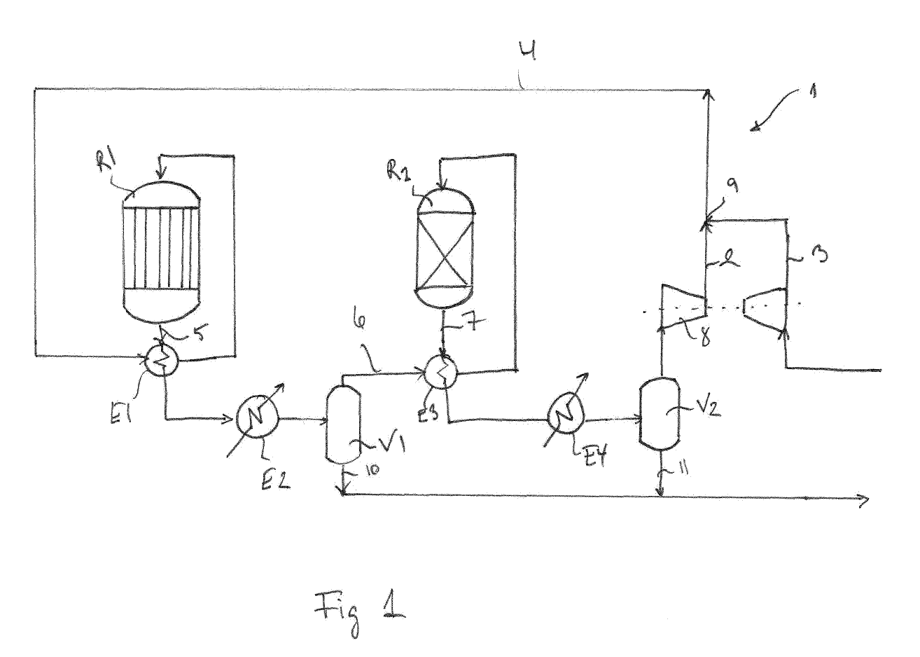

In FIG. 1 a schematic of the process and Methanol loop 1 is seen. Recycle gas 2 and Make-up gas 3 is mixed to a mixed stream 4. The Mixed stream consisting of the make-up gas (synthesis gas) and recycle is heated in E1 and introduced to the 1st Methanol reactor, R1, where the gas is converted over a Cu/Zn/Al2O3 catalyst (e.g. Topsøse MK-121 or MK-151 Fence™) The effluent 5 from the first reactor is cooled in E1/E2, and most of the produced methanol is condensed and separated in a gas/liquid separator, V1. The vapour phase (first unreacted stream) 6 is re-heated in E3 and introduced to the 2nd Methanol reactor, R2, where the gas is converted over a catalyst such as a 6×4 mm Cu/Zn/Al2O3 catalyst (e.g. Topsøse MK-121 or MK-151 Fence™). The effluent 7 from the second reactor gas is cooled in E3/E4 and condensed methanol is separated in the gas/liquid separator, V2. The gas from the second separator is returned to the recirculator compressor 8 and compressed and recycled to the mix point 9. The condensed methanol 10, 11 from the first and second separator is led to storage and/or further treatment.

### EXAMPLE: COMPARISON TO PRIOR ART

The present process is compared to prior art including WO2011101081,

### Example 1

Configuration as per the present process and plant, i.e. with condensation and separation of methanol between reactors

### Example 2

Configuration as per prior art i.e no separation between reactors

### Example 3

Configuration as per prior art, i.e. no separation between reactors, and with higher severity (lower inlet temperature) for the catalyst

The following feed gas to 1^{st} reactor has been used for all cases

1st Reactor Feed gas

| | | |
|---|---|---|
| Pressure | kg/cm2 g | 84.6 |
| Temperature | ° C. | 221 |
| Composition | mole-% | |
| H₂ | | 54.77 |
| CO | | 6.92 |
| CO₂ | | 7.34 |
| CH₄ | | 26.00 |
| N₂ | | 4.40 |
| CH₃OH | | 0.46 |
| H₂O | | 0.11 |
| Total | | 100.00 |

In the following table the conversion of CO/CO₂ into methanol is given. It is seen that the conversion is higher for example 1. In example 3 a higher conversion than in example 2 is obtained by a higher catalyst volume (or alternatively by a catalyst with higher activity) However, a conversion as high as for example 1 is not achieved.

| | Invention | | Prior art | | | |
|---|---|---|---|---|---|---|
| | Example 1 | | Example 2 | | Example 3 | |
| | 1st reactor | 2nd reactor | 1st reactor | 2nd reactor | 1st reactor | 2nd reactor |
| Type | BWR | Adiabatic | BWR | Adiabatic | BWR | Adiabatic |
| Outlet pressure, | 82.4 | 76.9 | 82.4 | 76.9 | 82.4 | 77.2 |
| kg/cm² g | | | | | | |
| Inlet | 221 | 221 | 221 | 221 | 221 | 200 |
| temperature, ° C. | | | | | | |
| Outlet | 253 | 242 | 253 | 242 | 253 | 230 |
| temperature, ° C. | | | | | | |
| CO/CO2 conversion | 34.0% | 18.4% | 34.0% | 8.0% | 34.0% | 11.2% |
| Relative catalyst | 100 | 100 | 100 | 100 | 100 | 173 |
| volume (relative | | | | | | |
| to catalyst volumes | | | | | | |
| in invention) | | | | | | |

1. A process comprising the steps of
providing a syngas stream with module M to a Methanol loop,
in the Methanol loop passing the syngas though a first Methanol reactor,
obtaining a first effluent from the first Methanol reactor,
cooling the first effluent and condensing at least part of the produced methanol
separating the first cooled effluent into at least a first raw Methanol stream and a first unreacted stream,
heating the first unreacted stream,
passing the first heated unreacted stream through a second methanol reactor,
obtaining a second effluent from the second methanol reactor, cooling and condensing said second effluent,
separating the cooled second effluent into at least a second raw Methanol stream and a second unreacted stream, and
recycling the second unreacted stream to the syngas stream.

2. A Process according to claim 1 wherein additional steps of conversion in a methanol reactor and methanol separation is applied.

3. A Process according to claim 1 wherein the first reactor and or second reactor is a boiling water reactor (BWR), an adiabatic reactor and/or a quench reactor.

4. A Process according to claim 1 wherein the first reactor and or second reactor is operated at the same inlet temperature.

5. A Process according to claim 1 wherein the first reactor and or second reactor is operated at different inlet temperatures.

6. A Process according to claim 1 wherein the first reactor and or second reactor operated with the same or different catalyst.

7. A Process according to claim 1 wherein the process is applied as part of a revamp.

8. A plant comprising one or more Methanol loops, said methanol loop comprising at least a first and second serially connected Methanol reactors wherein a first separator is arranged downstream the first Methanol reactor and upstream the second Methanol reactor and a second separator is arranged downstream the second reactor.

9. A plant according to claim 8 comprising one or more compressors, stream cooling means and stream heating means.

10. A plant according to claim 8 arranged to carry out the steps of:
providing a syngas stream with module M to the Methanol loop,
in the Methanol loop passing the syngas though the first Methanol reactor,
obtaining a first effluent from the first Methanol reactor,
cooling the first effluent and condensing at least part of the produced methanol,
separating the first cooled effluent into at least a first raw Methanol stream and a first unreacted stream,
heating the first unreacted stream,
passing the first heated unreacted stream through the second Methanol reactor,
obtaining a second effluent from the second methanol reactor, cooling and condensing said second effluent,
separating the cooled second effluent into at least a second raw Methanol stream and a second unreacted stream, and
recycling the second unreacted stream to the syngas stream.

11. A method for optimizing an existing methanol loop, said method comprising the steps of introducing the process according to claim 1.
